(19) United States
(12) Patent Application Publication
(10) Pub. No.: US 2006/0129212 A1
(43) Pub. Date: Jun. 15, 2006

(54) Method and apparatus for applying heat or cold to a body

(76) Inventors: Sarah Halvorson, Kaiser, OR (US); Doug Halvorson, Kaiser, OR (US)

Correspondence Address: HalSci Solutions, 998 Parkmeadow Drive N.E., Kaiser, OR, 97303, US

(21) Appl. No.: 11/304,297

(22) Filed: Dec. 15, 2005

Related U.S. Application Data
(60) Provisional application No. 60/636,171, filed on Dec. 15, 2004.

(51) Int. Cl. A61F 7/00

(52) U.S. Cl. 607/96

(57) ABSTRACT

An apparatus for applying heat and/or cold to a body and, in particular to a wearer's chest, and for allowing relatively easy application and removal of the device while further allowing for relative mobility of the wearer the apparatus having an elongated section having a outside side and an underside and including a first pocket attached away from the center of the elongated section toward a first end of the elongated section and a second pocket attached away from the center of the elongated section toward a second end of the elongated section and further having a first packet held in the first pocket, the first packet being capable of maintaining a temperature and a second packet held in the second pocket, the second packet capable of maintaining a temperature. Methods of utilizing the apparatus are described.

### RELATED APPLICATION(S)

This application claims benefit of a related application of the same title filed Dec. 15, 2004 which received U.S. Patent Office Ser. No. 60/636,171.

### DESCRIPTION

### BACKGROUND OF THE INVENTION

It is well known that the application of heat or cold to a body may have therapeutic effects. For example, see U.S. Pat. Nos. 5,050,595 titled Therapeutic Garment; 5,133,348 titled Contoured Cooling Pack; and 6,669,715 titled Medical Thermal Energy Exchange Pad.

The '348 patent is an example of a device that may be contoured to a body. Dependent on the part of a body to which it is applied, there may be varying degrees of success with applying the device while still allowing mobility of the body. For example, it can be seen that the device could be contoured around a knee or elbow in such a manner as to allow for mobility of the body. However, if the device is applied to the chest, mobility of the body will likely be restricted because movement of the body would cause displacement of the device.

It would be desirable to have a device that allows for application of heat or cold to the chest while still allowing for mobility of the body. The '595 patent provides a device that allows for application of heat to the chest while still allowing for relative mobility over the device of the '348 patent. However, the device of the '595 patent is relatively cumbersome to put onto the body and to remove.

It would be desirable to provide a device that is relatively easy to apply and remove from a body while still allowing for mobility.

### SUMMARY

An apparatus for applying heat and/or cold to a body and, in particular to a wearer's chest is described. The described apparatus allows for relatively easy application and removal of the device while further allowing for relative mobility of the wearer. The apparatus includes an elongated section having a outside side and an underside and includes a first pocket attached away from the center of the elongated section toward a first end of the elongated section and a second pocket attached away from the center of the elongated section toward a second end of the elongated section. The apparatus further includes a first packet held in the first pocket, the first packet being capable of maintaining a temperature and a second packet held in the second pocket, the second packet capable of maintaining a temperature.

Methods of utilizing the apparatus are described.

### BRIEF DESCRIPTION OF THE DRAWINGS

FIG. 1 is an illustration of person wearing an apparatus as may implement the present invention.

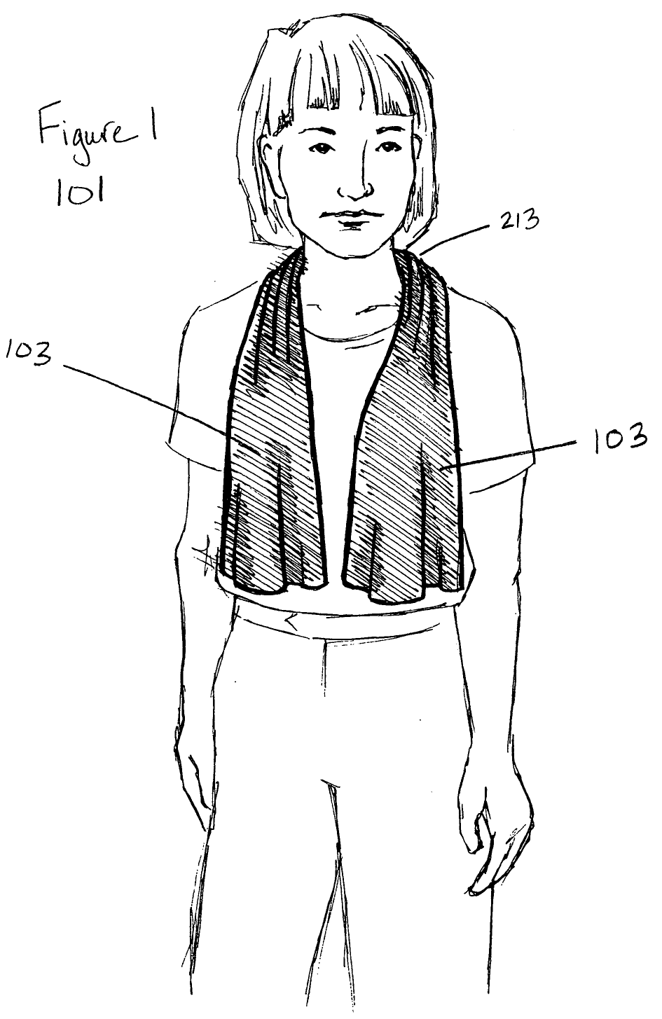

FIG. 2 is a second illustration of a person wearing an apparatus as may implement the present invention further illustrating the underside or second side of the apparatus.

FIG. 3 is a further illustration of certain details of an apparatus as may implement the present invention.

FIG. 4 is a flow diagram illustrating a method of utilizing an apparatus as may implement the present invention.

FIG. 5 is a flow diagram illustrating a second method of utilizing an apparatus as may implement the present invention.

### DETAILED DESCRIPTION

It is well known that application of heat or cold to a body has therapeutic effects. Ice packs, heating pads, gel packs and even bags of frozen peas are used to apply heat or cold to a body for many therapeutic reasons. For example, it is accepted that application of heat or cold to a body may prevent swelling; relieve pain; reduce discomfort from fever and, in fact, reduce fever; and allow for increased performance early in a weightlifting workout and reduce body heat following a weightlifting workout.

Many devices for applying heat or cold to a body are well known. Typically, these devices provide for applying heat or cold to an extremity (arm, leg). Often, the devices may be wrapped around the extremity and the person may, therefore, maintain some level of mobility even while benefiting from the therapeutic effects of the device. While these devices work well on extremities, when applied to the torso of a body, mobility is necessarily limited because there is typically no easy way to wrap the device around the torso.

Devices can be, and have been, designed which allow application of heat or cold to the torso of body while still providing for a level of mobility. One example is illustrated by U.S. Pat. No. 5,050,595. The '595 patent illustrates a “women's therapeutic support garment comprising a pair of breast supporting cups each of which is formed with an inner and outer panel defining there between one of two cup shaped packets.” It is clear from a review of the '595 patent that putting on or taking off a device as described in the '595 patent requires removal of the wearer's outer garments (e.g., shirt or blouse) and removal of the device itself fully exposing the wearer's body. This not only has potential for being time consuming but also for being relatively cumbersome while still allowing for some degree of privacy for the wearer.

Thus, as has been stated, it is highly desirably to develop a device that both allows for mobility and can be applied or removed with minimal time and intrusion.

An embodiment of the present invention involves an elongated section 103 worn about the wearer's neck. In one form, the present invention advantageously may be viewed by the observer as a scarf and, thus, may be worn relatively discretely in many situations by the wearer 101 in many situations. In fact, as will be described, in one embodiment of the invention, the outside (or first side) of the elongated section is manufactured using an upholstery grade fabric. As will be appreciated, use of a relatively heavy weight fabric on the outside of the elongated section allows the for the device to “drape” in a manner that the pockets and packets which will be described in further detail below are relatively hidden from view.

As can be readily appreciated, such a device 103 as is described and illustrated may be easily put on or taken off without any putting the wearer 101 in any uncompromising position.

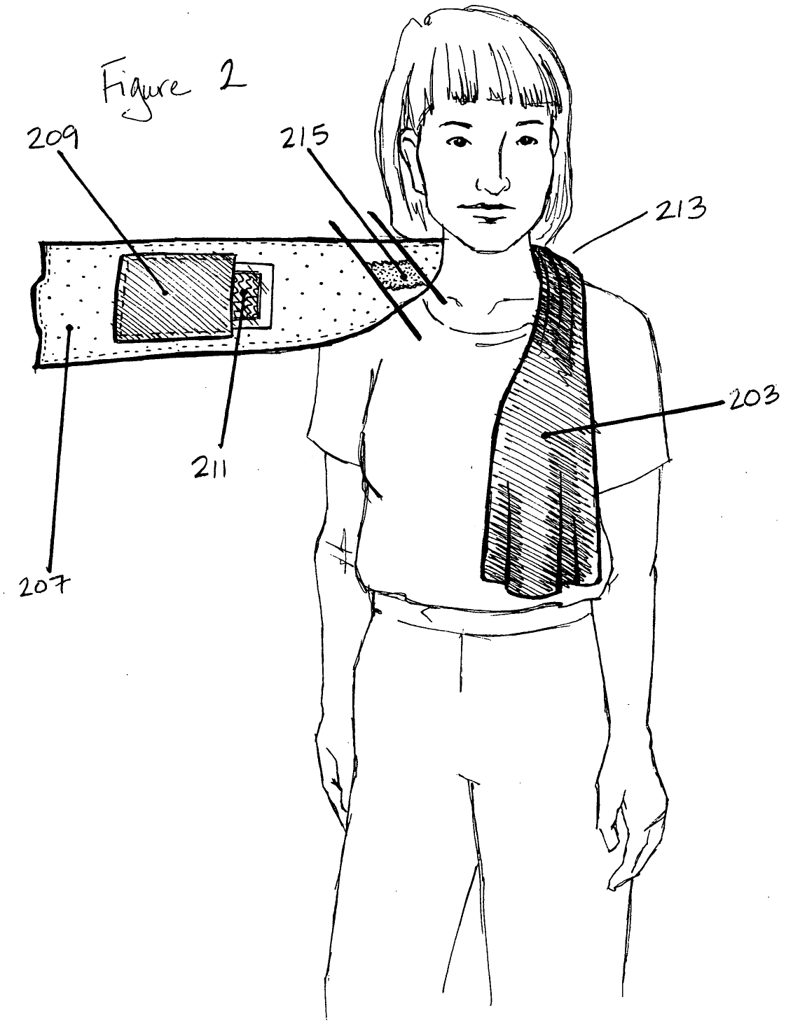

FIG. 2 illustrates the elongated section 103 in further detail. As shown in FIG. 2, the elongated section 103 includes the aforementioned first side 203 and an underside (the second side) 207. While any of a number of materials may be used for the underside 207, in one embodiment, a material which is relatively softer and smoother than the material used for the first side 203 is chosen. The softer and smoother material may comprise, for example, a polyester material. Use of a softer and smoother material is advantageous because the underside 207 makes contact with the wearer's body for example especially about the neck 213.

FIG. 2 further illustrates a first pocket 209 attached to the elongated section 103. In the illustrated example, the pocket 209 is attached to the underside 207; however, in alternative embodiments, the pocket may be attached to the first side 203. Advantageously, attaching the pocket 209 to the underside 207 allows the pocket 209 to be relatively hidden from view. The pocket 209 is attached away from the center of the elongated section 103 toward a first end of the elongated section 203. In the described embodiment, the pocket 209 is attached at a position such that when the elongated section 103 is worn with a middle portion of the elongated section 103 about the wearer's neck 213, the pocket 209 hangs above one side of the wearer's chest.

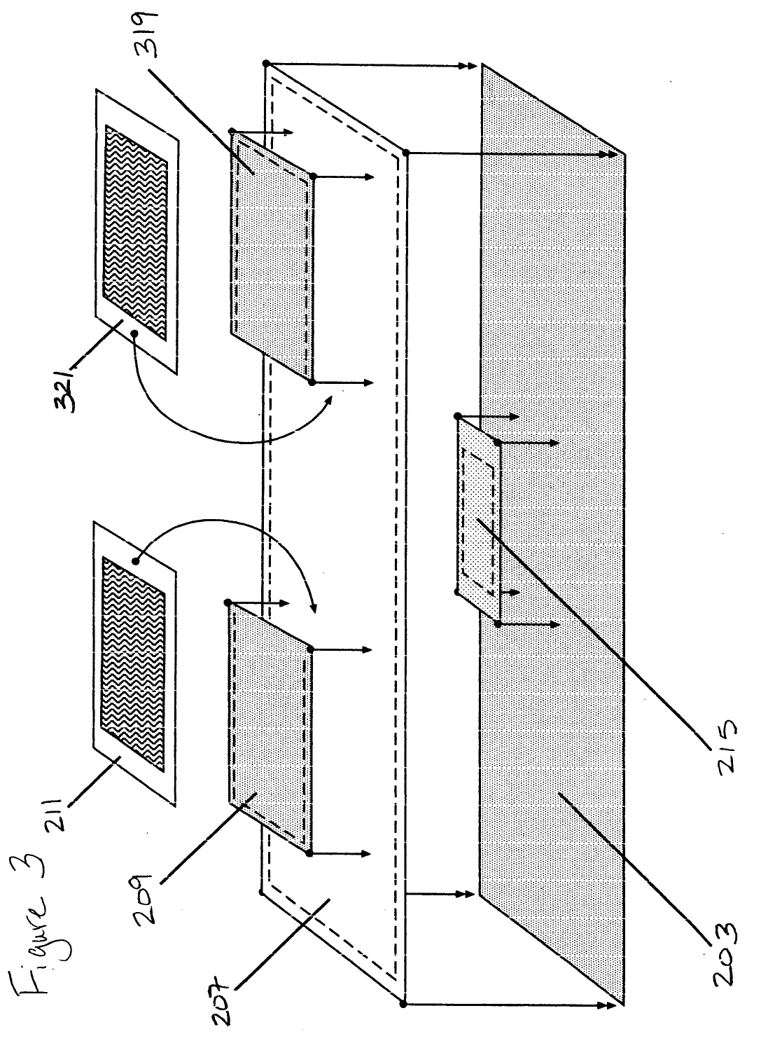

As will be seen from FIG. 3, a second pocket 319 is attached to the elongated section 103 toward its second end. Thus, when worn about the wearer's neck 213, the first pocket 209 hangs about one side of the wearer's chest and the second pocket 319 hangs about the other side of the wearer's chest.

Pocket 209 holds packet 211. Packet 211 is capable of maintaining a temperature (e.g., hot or cold) and may compromise any of a number of materials. For example, packet 211 may contain simply any material which is capable of maintaining a temperature such as a something as simple as packet of frozen vegetables. In addition, there are numerous commercially available products which are designed to maintain a temperature which typically take the form of a packet enclosing some form of gel. Preferably, the packet provides the following characteristics:
stays hot or cold for 20-40 minutes;
may be heated in a microwave or conventional oven;
may be cooled in a freezer;
remains soft and flexible when cold;
does not leak even if the packet is punctured.

One commercially available product which provides for a number of these characteristics is the Elasto-Gel™ hot/cold therapy products available from Southwest Technologies, Inc. of North Kansas City, Mo.

Advantageously, in one embodiment, packet 211 may be removable from pocket 209. This allows the packet 211 to be heated or cooled independent of the elongated section 103 and, further allows replacement of the packet 211 with a new or different packet if replacement becomes desirable. However, alternative embodiments may permanently affix packet 211 into pocket 209.

Packet 321 typically would be of the same type and material as packet 211.

In one embodiment, the middle elongated section 103 includes a middle section 215 which comprises padding. The use of padding about the middle section 215 provides for increased comfort for the wearer when the elongated section 103 is worn about the neck 213. While many materials may be utilized, because of the comfort provided, one embodiment utilizes a polyfill material for the padding. Alternatively, in another embodiment, fleece is utilized because of its durability especially through washings. Alternatively, a combination of fleece and polyfill may be utilized.

While using padding in middle section 215 provides for comfort, certain applications of the elongated section 103 may benefit from providing a third pocket at the middle section 215 and a third packet for insertion into the packet so that heat or cold may also be applied to the wearer's neck 213.

As can now be readily seen, utilizing elongated section 103, a wearer may easily apply heat or cold to the chest with a device which may be easily put on or taken off and which may be worn in many social situations without embarrassment. In one application, it is readily seen that the use of the described device allows for application of heat or cold to the wearer's chest. As has been discussed, application of heat or cold is known to have therapeutic effects. Use of the above described device may be particularly useful for persons recovering from injury or surgery to the chest and for women during the premenstrual period, during pregnancy or during the post-partum period.

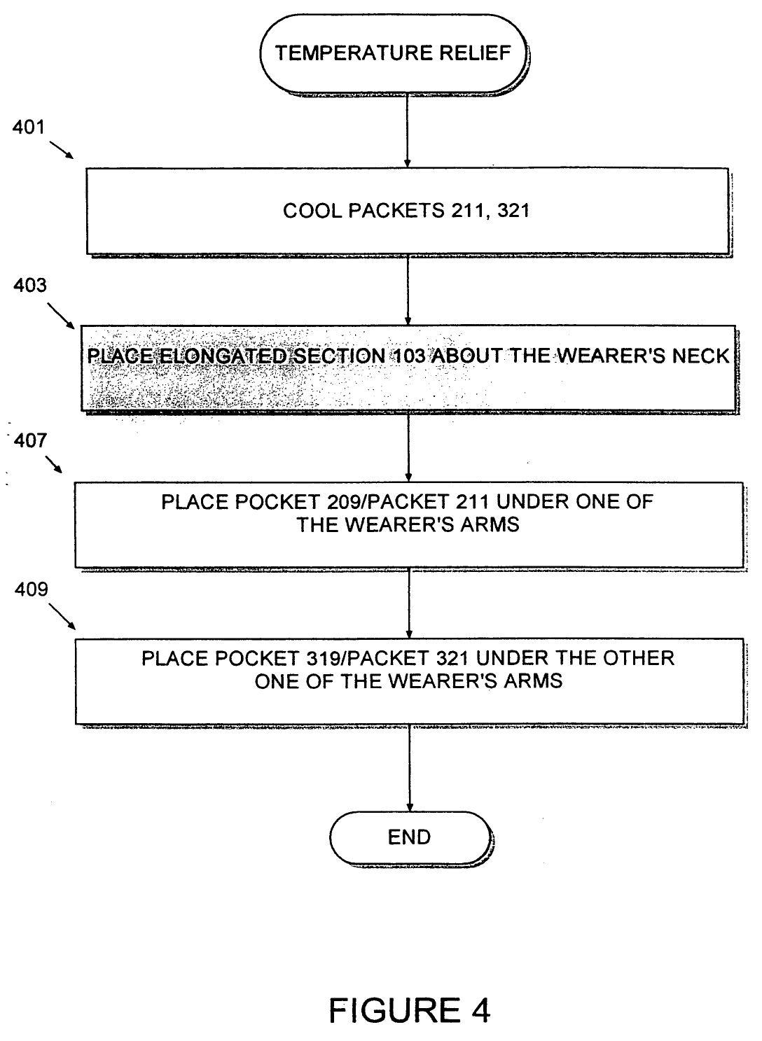

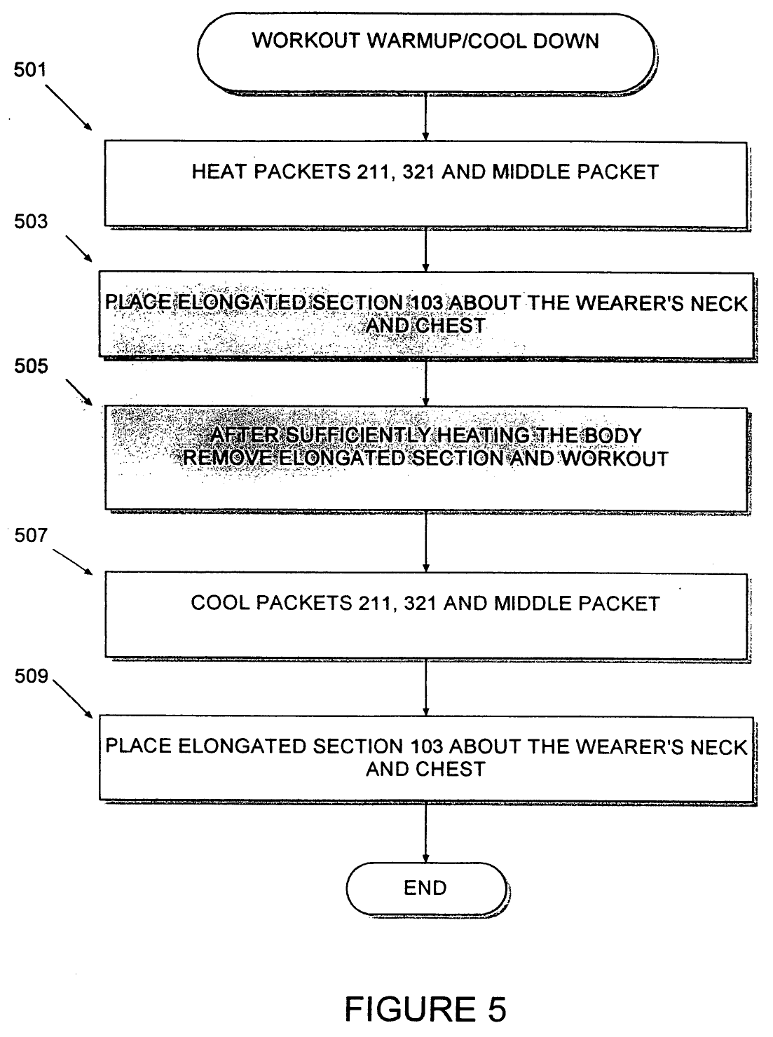

There are, of course, many other applications of the described device. Without limitation to the wide variety of applications of the device, FIGS. 4 and 5 provide further explanation of two uses of the device.

Turning first to FIG. 4, it has been established that applying a cold temperature under the arms can help to control temperature in the body during periods of fever and to alleviate symptoms of the fever. The described device is useful for applying cold under the arms. As is described, first packets 211 and 321 (and the third packet used in the middle section, if provided) are cooled, step 401. The elongated section 103 is then placed about the wearer's neck, step 403. Pocket 209/packet 211 are then placed under one of the wearer's arms, step 407 and pocket 319/packet 321 are placed under the other of the wearer's arms, step 409.

In another application, prior to engaging in a workout such as a weight workout in a gym, typically, the person will want to do some warm-up exercises to allow the muscles to stretch out in order to avoid or minimize injury. Unfortunately, all too often, time available for a workout is limited. It is established that application of heat to muscles prior to a workout may shorten or eliminate the need for a warm-up. Thus, turning to FIG. 5, prior to a workout, a user may heat the packets 211, 321 and, if available, the middle packet, step 501 and place the elongated section 103 about the user's neck and chest, step 503. The user may, for example, heat the packets and apply the device before leaving home to go to a gym and leave the device in place during the drive to the gym. Thus, when arriving at the gym, the user's chest (and neck, if the middle packet is available and used) are warmed. When arriving at the gym, the device can be removed and the user can workout, step 505.

Advantageously, the device can also be used to alleviate another problem often faced by people doing workouts. Following the workout, the person is typically hot and cooling down takes time; and, again, time is often not available to the user. Thus, the wearer may cool packets 211, 321 and, if available, the middle packet, step 507. Preferably, the wearer has purchased a second set of packets so that one set can be warmed and used prior to the workout as has been described and a second set can be cooled and kept cool during the workout so that they are available after the workout. Then, following the work, step 505, the cooled packets can be inserted into elongated section 103 and the device can be worn about the wearer's chest, step 509.

The present invention has been described with reference to specific embodiments. However, the invention is intended to be limited and defined by the claims below and not by any specific embodiment described herein.

What is claimed is:

1. An apparatus comprising:
an elongated section having a first side and a second side;
a first pocket attached away from the center of the elongated section toward a first end of the elongated section;
a second pocket attached away from the center of the elongated section toward a second end of the elongated section;
a first packet held in the first pocket, the first packet being capable of maintaining a temperature; and
a second packet held in the second pocket, the second packet capable of maintaining a temperature.
The apparatus as recited by claim 1 wherein the packets are attached to the second side of the elongated section.
The apparatus as recited by claim 1 wherein the first side is comprised of an upholstery grade fabric.
The apparatus as recited by claim 1 wherein the second side is comprised of a softer fabric than the first side.
The apparatus as recited by claim 1 wherein the second side is comprised of a polyester fabric.
The apparatus as recited by claim 1 wherein a middle section of the elongated members comprises padding.
The apparatus

9. The apparatus as recited by claim 1 wherein the first packet maintains heat.

10. The apparatus as recited by claim 1 wherein the first packet maintains cold.

11. The apparatus as recited by claim 1: wherein the first packet remains flexible and soft at low temperatures.

12. The apparatus as recited by claim 1 wherein the first packet comprises Elasto-Gel™.

13. The apparatus as recited by claim 1 wherein a middle section of the elongated members comprises a third pocket and said third pocket holds a third packet.

14. An scarf for wearing about a neck and for providing therapeutic effects of heat and cold to the wearer's chest, the scarf comprising:
a) a first side and a second side;
b) a first pocket attached away from the center and toward a first end of the scarf;
c) a second pocket attached away from the center and toward a second end of the scarf;
d) a first packet held in the first pocket, the first packet being capable of maintaining a temperature; and
e) a second packet held in the second pocket, the second packet capable of maintaining a temperature.

15. The scarf as recited by claim 14 wherein the packets are attached to the second side of the elongated section.

16. The scarf as recited by claim 14 wherein the first side is comprised of an upholstery grade fabric.

17. The scarf as recited by claim 14 wherein the second side is comprised of a softer fabric than the first side.

18. The scarf as recited by claim 14 wherein the second side is comprised of a polyester fabric.

19. The scarf as recited by claim 14 wherein a middle section of the scarf comprises padding.

20. The scarf as recited by claim 19 wherein the padding comprises fleece.

21. The scarf as recited by claim 19 wherein the padding comprises polyfill.

22. The scarf as recited by claim 14 wherein the first packet maintains heat.

23. The scarf as recited by claim 14 wherein the first packet maintains cold.

24. The scarf as recited by claim 14 wherein the first packet remains flexible and soft at low temperatures.

25. The scarf as recited by claim 14 wherein the first packet comprises Elasto-Gel™.

26. The scarf as recited by claim 14 wherein a middle section of the scarf comprises a third pocket and said third pocket holds a third packet.

27. A scarf have a first end and a second end including a first apparatus disposed toward the first end of the scarf capable of maintaining a temperature and a second apparatus disposed toward a second end of the scarf capable of maintaining a temperature.

28. The scarf as recited by claim 27 further comprising a first pocket disposed toward the first end of the scarf for holding the first apparatus and a second pocket disposed toward the second end of the scarf for holding the second apparatus.

29. The scarf as recited by claim 28 wherein the first pocket removably holds the first apparatus.

30. The scarf as recited by claim 27 wherein the first apparatus comprises a gel packet.

31. The scarf as recited by claim 27 wherein the scarf has a first side and a second side, said first side being comprised of a relatively heavier fabric than said second side.

32. A method of warming up muscles prior to a workout comprising:
a) heating packets capable of maintaining a temperature;
b) applying heat from the packets to a user's chest by wearing a scarf containing the packets and positioning the packets over the user's chest;
c) removing the scarf and commending a workout after the user's muscles have been sufficiently warmed.

33. The method as recited by claim 32 further including steps providing for cooling the muscles after the workout comprising:
a) cooling packets capable of maintaining a temperature;
b) applying coolness from the packets to a user's chest by wearing a scarf containing the packets and positioning the packets over the user's chest;
c) removing the scarf after the user's muscles have been sufficiently cooled.
